(19) United States
(12) Patent Application Publication
(10) Pub. No.: US 2016/0206788 A1
(43) Pub. Date: Jul. 21, 2016

(54) BIOMIMETIC INTERFACES FOR BIODEGRADABLE METALLIC IMPLANTS

(71) Applicant: THE REGENTS OF THE UNIVERSITY OF CALIFORNIA, Oakland, CA (US)

(72) Inventor: HUINAN LIU, RIVERSIDE, CA (US)

(73) Assignee: THE REGENTS OF THE UNIVERSITY OF CALIFORNIA, OAKLAND, CA (US)

(21) Appl. No.: 14/303,321

(22) Filed: Jun. 12, 2014

Related U.S. Application Data
(60) Provisional application No. 61/834,077, filed on Jun. 12, 2013.

(51) Int. Cl. A61L 31/02; A61L 31/10; A61L 31/14; A61L 31/08

(52) U.S. Cl. CPC A61L 31/022; A61L 31/086; A61L 31/10; A61L 31/148

(57) ABSTRACT

A biodegradable device includes a substrate and a nano-ceramic/polymer composite coating thereon. The substrate may include MgY, AZ alloys, MgCa, MgZn, MgZnCa, MgZnCaZr, MgZnSr, MgLi, Ti, Ti-6Al-4V, CoCr, and CoCrMo. The composite coating may include a nano-ceramic and a polymer.

### STATEMENT OF GOVERNMENT INTEREST

None.

### BACKGROUND

The present invention relates to biodegradable metal implants and, more specifically, magnesium-based metallic implants and their surfaces.

Millions of screws, pins, plates and suture anchors are used for internal fixation of bone fracture and surgical repair and reconstruction of soft tissues in orthopedic and maxillofacial surgeries annually. Despite their widespread applications, the choice of materials for these surgical fixation devices traditionally has been limited to non-degradable metals, like stainless steel and titanium (Ti) alloys, or bioabsorbable polymers, like poly-L-lactic acid (PLLA), poly-glycolic acid (PGA) and their copolymers, or biocomposites. Ti alloys offer a good initial fixation strength due to their inherent high modulus and strength and provide acceptable cytocompatibility due to their bio-inertness. However, their permanency is problematic for pediatric patients, leading to growth restriction, intracranial migration, skeletal alteration and other irregularities. Thus, subsequent surgeries are often needed for their removal.

Moreover, steel and Ti alloys can release toxic metallic ions and/or particles, and their elastic moduli are not well matched with that of natural bone, resulting in stress shielding effects on healing tissue which decrease implant stability in the long term. In addition, these metals distort post-operative evaluation by magnetic resonance imaging (MRI).

Alternatively, bioabsorbable polymeric screws, plates and suture anchors have been developed in recent years to obviate the need for their removal. However, device breakage occurs frequently due to their low modulus and strength (generally 10 times lower than those for Ti alloys). Many have reported other complications, including poor osseointegration and highly variable degradation, ranging from almost complete degradation within one year to a significant presence at four years post-operatively.

Recently, biocomposite materials composed of polyester combined with ceramic particles (e.g., hydroxyapatite or β-tricalcium phosphate) have been introduced to address the poor osseointegration of the bioabsorbable polymeric screws. But device breakage during and after surgery remains a significant problem due to the inherent bulk properties of the polymer matrix.

Because of clinical problems associated with these traditional materials, a class of biodegradable metallic materials, i.e., magnesium-based alloys, has been actively pursued. Magnesium (Mg) has beneficial properties for bone regeneration and its osteoconductivity has been shown in vitro and in vivo. The elastic modulus and mechanical strength of Mg are similar to cortical bone (Table 1), which reduces the stress-shielding effects of implants on surrounding bone and makes Mg desirable for load-bearing implant applications. Moreover, Mg degrades in physiological environments, mainly through reactions with water in the body fluids, as described in the following reactions. Mg degradation in the body fluids eventually produces Mg cations (Mg²⁺), hydroxide anions (OH⁻), and hydrogen gas (H₂). Kidneys in the body excrete Mg ions efficiently and eliminate them naturally through urine if the concentration is within the tolerable range.

Mg+2H₂O→Mg(OH)₂+H₂↑ (1)

Mg→Mg²⁺+2e⁻ (1a)

2H₂O+2e⁻H₂↑+2OH⁻ (1b)

Mg²⁺+2OH⁻←→Mg(OH₂)↑ (1c)

Biodegradability of Mg is advantageous and desirable for medical implants that only serve temporary functions during tissue healing, such as fixation devices for bone fracture and soft tissue repair (e.g., reconstruction of anterior cruciate ligament, repair of shoulder rotator cuff). After tissue has healed, these implants are no longer needed in the body and are often surgically removed. In comparison, the use of implants made of biodegradable materials could eliminate the need for surgical removal, thus reducing associated clinical complications and healthcare cost.

As Mg degrades rapidly, however, OH⁻ and H₂ accumulate locally and adversely affect surrounding cells and tissues. Specifically, OH⁻ions cause the increase of local pH, which may be harmful to cells. Rapid degradation of Mg also reduces the mechanical strength of implants before the healing tissue regains its strength, which may cause catastrophic failure of the implants prematurely. Therefore, it is necessary to moderate the degradation of Mg and its alloys in order to satisfy the clinical requirements for these implants.

The degradation rate of Mg-based materials can be controlled by surface treatment and/or modification of alloy composition and processing parameters. It has been reported that the degradation rate decreased to some extent through careful selection of Mg with high purity to eliminate internal galvanic reactions, or through the addition of certain alloying elements. Production of highly pure Mg requires expensive purification processes, while the alloying approach involves elements that may increase the risk of cytotoxicity. Controlling the interface of magnesium with the biological environment is a key challenge that currently limits this biodegradable metal for broad applications in medical devices and implants.

Accordingly, there is a need for a surface treatment strategy, specifically using a functional nanocomposite coating, to control Mg degradation and to achieve other synergistic properties.

### SUMMARY

In one aspect of the invention, a biodegradable device comprises a substrate made of a biodegradable metal selected from the group consisting of MgY, AZ alloys, MgCa, MgZn, MgZnCa, MgZnCaZr, MgZnSr, MgLi, and a nano-ceramic/polymer composite coating on the substrate.

In another aspect of the invention, a medical device comprises a substrate made of a metal selected from the group consisting of Ti, Ti-6Al-4V, CoCr, and CoCrMo; and a nano-ceramic/polymer composite coating on the substrate.

In a further aspect of the invention, a biodegradable device comprises a metallic substrate; and a nano-ceramic/polymer composite coating on the substrate, wherein the coating includes a nano-ceramic and a polymer.

These and other features, aspects and advantages of the present invention will become better understood with reference to the following drawings, description and claims.

### BRIEF DESCRIPTION OF THE SEVERAL VIEWS OF THE DRAWINGS

Table 1 shows properties of Mg, HA, and PLGA in comparison with cortical bone;

FIG. 1 is an illustration of the interaction among nHA, PLGA, and Mg substrates according to the present invention;

FIG. 2A is an SEM image of synthesized nHA particles according to the present invention;

FIG. 2B is a binary mask of the SEM image of FIG. 2A;

FIG. 2C is an EDX spectrum of the nHA according to the present invention;

FIG. 2D is an XRD spectrum of the nHA according to the present invention;

FIG. 3A is an SEM image of a PLGA coated Mg substrate;

FIG. 3B is an SEM image of a nHA/PLGA coated Mg substrate;

FIGS. 4A-4H are SEM images of surfaces of materials according to the present invention;

FIG. 5A shows Tafel plots of studied materials according to the present invention;

FIG. 5B shows predicted Mg corrosion rates of studied materials according to the present invention;

FIG. 6 shows corrosion potentials of studied materials according to the present invention;

FIG. 7 show photographs of studied materials after immersion in rSBF according to the present invention;

FIGS. 8A-8D show plots of mass change of coated and non-coated studied materials according to the present invention;

FIGS. 8A′-8D′ show plots of pH change of coated and non-coated studied materials according to the present invention;

FIG. 9 show plots of ion concentration in rSBF of studied materials according to the present invention;

FIGS. 10A-10C are SEM images of studied materials after degradation in rSBF according to the present invention;

FIGS. 10A′-10C′ are EDX analyses of studied materials after degradation in rSBF according to the present invention;

FIGS. 11A-11L show hydrogen gas evolution of studied materials after degradation in rSBF according to the present invention;

FIG. 12 is an illustration of degradation process of studied materials according to the present invention;

FIG. 13 is an illustration of an embodiment of the present invention.

### DETAILED DESCRIPTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, embodiments of the present invention provide coatings of nanocomposites on substrates that meet mechanical, degradation, and tissue integration requirements for a wide range of orthopedic, maxillofacial and pediatric surgery use (Exhibit 13). The nanocomposite coatings herein provide a novel surface treatment solution for metallic implants and make a wide range of medical applications of alloys possible. The nanocomposites herein provide a biomimetic interface between tissue and implants, more advantageous compared to current calcium phosphate coating.

The substrates can be of any geometry. Exemplary embodiments include orthopedic implants, craniomaxillofacial implants, surgical sutures, staples, cardiovascular implants/stents, embolic coils for aneurysm treatment, and urological devices (e.g., stents and catheters).

In exemplary embodiments, the substrate may be made of magnesium alloys, for example, MgY, AZ alloys, MgCa, MgZn, MgZnCa, MgZnCaZr, MgZnSr and MgLi.

In exemplary embodiments of MgY, Y may be present from about 0.01 to about 1 wt. % of the alloy, or from about 1 to about 2 wt. % of the alloy, or from about 2 to about 4 wt. % of the alloy, or from about 4 to about 8 wt. % of the alloy.

In exemplary embodiments of AZ alloys, Al may be present from about 0.01 to about 1 wt. % of the alloy, or from about 2 to about 5 wt. % of the alloy, or from about 6 to about 12 wt. % of the alloy; and, respectively, Zn may be present from about 0.1 to about 1 wt. % of the alloy, or from about 2 to about 6 wt. % of the alloy, or from about 6 to about 9 wt. % of the alloy.

In exemplary embodiments of MgCa, Ca may be present from about 0.01 to about 1.0 wt. % of the alloy, or from about 2 to about 5 wt. % of the alloy, or from about 6 to about 15 wt. % of the alloy.

In exemplary embodiments of MgZn, Zn may be present from about 0.01 to about 1.0 wt. % of the alloy, or from about 2.0 to about 4.0 wt. % of the alloy, or from about 4.5% to about 9.0 wt. % of the alloy.

In exemplary embodiments of MgZnCa, Zn may be present from about 0.01 to about 1.0 wt. % of the alloy while Ca may be present from about 0.01 to about 1.0 wt. % of the alloy. In other embodiments, Zn may be present from about 1.0 to about 4.0 wt. % of the alloy while Ca may be present from about 0.01 to about 1.0 wt. % of the alloy. In further embodiments, Zn may be present from about 4.5 to about 9.0 wt. % of the alloy while Ca may be present from about 0.01 to about 1.0 wt. % of the alloy.

In exemplary embodiments of MgZnCaZr, Zn may be present from about 0.01 to about 1.0 wt. % of the alloy, Ca may be present from about 0.01 to about 1.0 wt. % of the alloy, and Zr may be present from about 0.01 to about 1.0 wt. % of the alloy. In other embodiments, Zn may be present from about 2.0 to about 4.0 wt. % of the alloy, Ca may be present from about 0.01 to about 1.0 wt. % of the alloy, and Zr may be present from about 0.01 to about 1.0 wt. % of the alloy. In further embodiments, Zn may be present from about 4.5 to about 9.0 wt. % of the alloy, Ca may be present from about 0.01 to about 1.0 wt. % of the alloy, and Zr may be present from about 0.01 to about 1.0 wt. % of the alloy.

In exemplary embodiments of MgZnSr, Zn may be present from about 2.0 to about 5.0 wt. % of the alloy while Sr may be present from about 0.01 to about 2.0 wt. % of the alloy. In other embodiments, Zn may be present from about 5.5 to about 9.0 wt. % of the alloy while Sr may be present from about 0.01 to about 2.0 wt. % of the alloy. In further embodiments, Zn may be present from about 0.1 to about 1.9 wt. % of the alloy while Sr may be present from about 2 to about 4 wt. % of the alloy, or Zn may be present from about 2 to about 7 wt. % of the alloy of the alloy while Sr may be present from about 2 to about 4 wt. % of the alloy.

In exemplary embodiments of MgLi, Li may be present from about 0.01 to about 1.0 wt. % of the alloy, or from about 1 to about 4 wt. % of the alloy, or from about 4% to about 6 wt. % of the alloy.

In other exemplary embodiments, the substrate may be commercially pure Ti, Ti-6Al-4V alloys, CoCr alloys, and CoCrMo alloys.

For example, in Ti6Al4V alloys, Ti may be present from about 85 to about 90 wt. % of the alloy, or from about 90 to about 95 wt. % of the alloy, while Al may be present from about 4 to about 6 wt. % of the alloy, and while V may be present from about 3 to about 5 wt. % of the alloy.

For example, in CoCrMo alloys, Cr may be present from about 27 to about 30 wt. % of the alloy, while Mo may be present from about 5 to about 7 wt. % of the alloy, and the rest is Co.

More specifically, embodiments of the present invention generally provide a nano-ceramic/polymer composite coating on the substrate to control substrate degradation in physiological environment. In exemplary embodiments, the coating may include one or more nano ceramics and one or more polymers. As an example, the nano ceramic may be from about 1 to about 30 wt %, or from about 1 to about 5 wt. %, or from about 5 to about 10 wt. %, or from about 10 to about 15 wt. %, or from about 15 to about 20 wt. %, or from about 20 to about 25 wt. %, or from about 25 to about 30 wt. %. In such embodiments, the remainder of the coating could be polymer.

In certain embodiments, the nano-ceramic of the composite coating may be nanostructured hydroxyapatite (nHA), nano MgO, nano ZnO, nano iron oxide, and nano TiO₂. Examples of the polymer in the composite coating may include poly(lactic-co-glycolic acid)(PLGA), poly lactide (also called poly lactic acid)(PLA), poly glycolide (also called poly glycolic acid)(PGA), polycaprolactone (PCL), PCL-PLGA (which is a copolymer of PCL and PLGA), PGS (polyglycerol sebacate). PLGA is copolymer of PLA and PGA. PLGA includes all derivatives of PLA, PGA, with a PLA/PGA ratio from 10/90, 20/80, 30/70, 40/60, 50/50, 60/40, 70/30, 80/20 to 90/10.

According to exemplary embodiments, the composite coating may have a thickness of about 50 nm to about 100 nm, or from about 100 nm to about 250 nm, or from about 250 nm to about 500 nm, or from about 500 nm to about 2 micrometer, or from about 2 micrometer to about 20 micrometer, or from about 20 micrometer to about 60 micrometer, or from about 60 micrometer to about 100 micrometer. These exemplary thicknesses might exist, irrespective of the thickness of the substrate, shape of the substrate, or amount of alloy in the substrate.

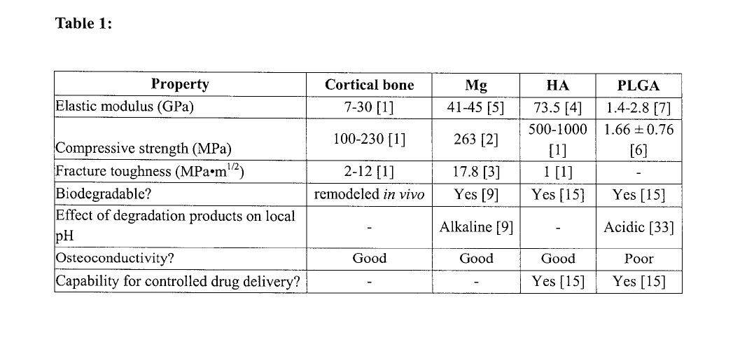

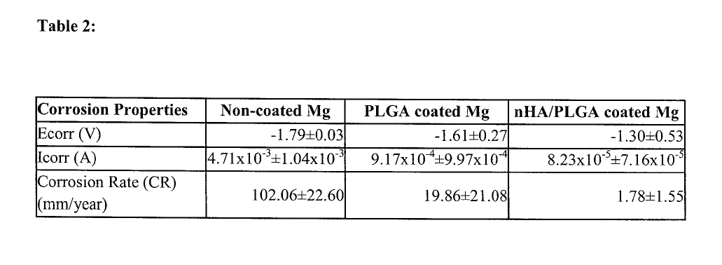

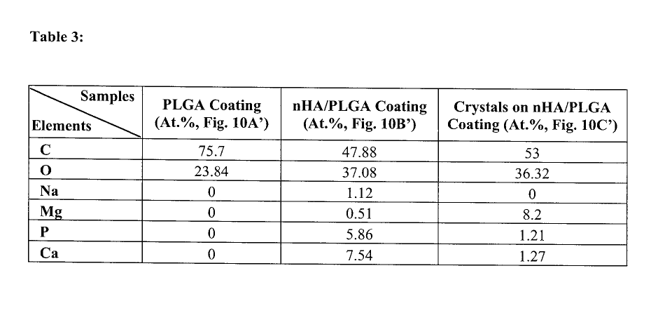

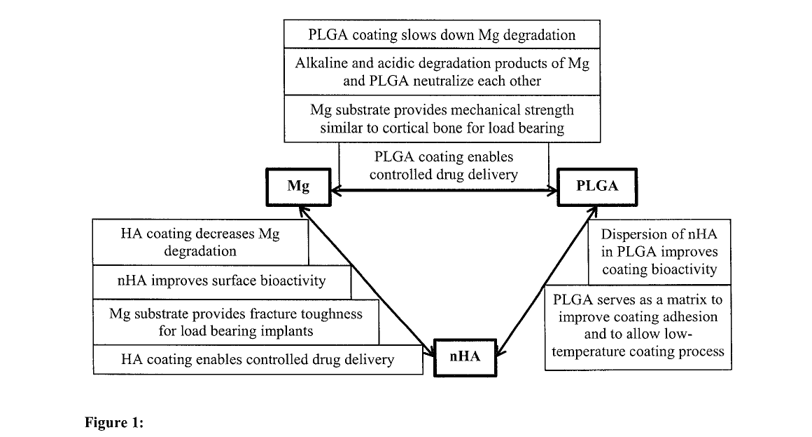

A novelty of the present coating design lies in the synergy among nHA, PLGA and Mg due to their complementary properties to one another, as illustrated in FIG. 1. That is, Mg and its alloys serve as biodegradable substrate materials to provide ideal mechanical properties for load-bearing implants, while nHA/PLGA composites serve as coating materials to control Mg degradation rate and improve osteointegration at the implant-tissue interface. The specific mechanical and biological properties of Mg, HA and PLGA in comparison with cortical bone are summarized in Table 1. The nHA/PLGA coated Mg provides desirable mechanical properties, enhanced osteoconductivity, and capability for controlled drug delivery, in addition to the fact that all three components (nHA, PLGA, and Mg) are biodegradable and biocompatible in the body. Moreover, the acidic degradation products of PLGA neutralize alkaline degradation products of Mg. More importantly, nanostructured HA improves surface bioactivity and osteoconductivity, and increases the deposition of calcium-containing bone minerals (e.g. calcium phosphates).

PLGA is one of the few biodegradable polymers that are approved by the Food and Drug Administration (FDA) for use in biomedical implants. PLGA degrades by reacting with water and releases acidic intermediate degradation products (i.e., lactic acid and glycolic acid) that are metabolized and removed from the body in the form of water and carbon dioxide. A PLGA scaffold can serve as a temporary extracellular matrix for cell growth and act as a vehicle for controlled drug delivery as PLGA is generally considered to be biocompatible. PLGA by itself lacks sufficient mechanical strength for load-bearing implant applications. PLGA coating has been investigated for controlling Mg degradation. A drawback is that PLGA has low osteoconductivity. Dispersing nanophase hydroxyapatite (nHA) in PLGA matrix provides a promising solution for enhancing osteointegration, while regulating Mg degradation simultaneously.

Nanophase HA (nHA), a derivative of calcium phosphates (CaP), promotes osteoconductivity and tissue-implant integration due to its similar chemistry and nano-scale features to natural bone minerals. HA can also serve as a drug carrier for controlled drug delivery. However, HA is too brittle to be used alone as a load bearing material. It has been reported that HA coatings improved Mg degradation resistance. However, directly coating HA alone onto metallic substrates causes several problems, dependent on the type of coating processes and the specific procedure used. High temperature coating processes, such as widely used plasma spray, transform HA into a different phase of calcium phosphates, alter the crystallinity of HA, or increase the grain size of HA from nano-scale to micron-scale. In comparison, HA coating deposited via low temperature processes, such as electrodeposition, typically has weaker adhesion strength without heat treatment. It is also noteworthy that HA delamination still occurred even if high temperature processes were used. A mismatch of thermal expansion coefficient between HA and the metallic substrate is one of the major reasons. Combining nHA with PLGA matrix as a coating material potentially circumvents the problems related to HA coating. Moreover, nHA/PLGA composites increase human mesenchymal stem cell adhesion and osteogenic differentiation, thus promoting bone regeneration.

Therefore, in the present invention, nanophase HA and PLGA composites are used as the coating material due to their biodegradability, biocompatibility, long history of FDA approved medical use, and potential synergistic advantages with Mg substrates as illustrated in FIG. 1. The use of nHA/PLGA coatings on Mg substrates is synergistic and beneficial due to their complementary properties (FIG. 1). The Mg substrate provides the mechanical strength and fracture toughness needed for load bearing applications. The nHA/PLGA coating controls Mg degradation and enhances cellular interactions at the tissue-implant interface. Nanophase HA dispersed in a PLGA matrix significantly improves osteoconductivity. The alkaline and acidic degradation products of Mg and PLGA neutralize each other, minimizing the local pH change. The use of PLGA as a matrix also makes room-temperature spin coating possible, thus addressing the problems (such as grain growth and phase change of HA) associated with current HA coating processes. Additionally, the nHA/PLGA composite coating enables controlled drug delivery to the tissue-implant interface. Synergy between the nanocomposite coatings and Mg substrates lead to a biodegradable implant with a multifunctional interface for more effective bone regeneration.

### Examples

### Materials and Methods

### Preparation and Characterization of Mg Substrates and the Controls

Commercially pure Mg, AZ31 alloy, and commercially pure titanium (Ti) were prepared as the substrates for coating processes. Specifically, a 1-mm thick Mg plate (99% purity; Miniscience), a 1-mm thick AZ31 plate (Alfa Aesar), and a 0.5-mm thick Ti plate (Alfa Aesar) were ground sequentially using 600, 800, and 1200 grit silicon carbide abrasive papers (Ted Pella) to remove the oxide layers on the surface. Ethanol (200 proof; Koptec) was used to lubricate the plates during grinding and washing away particulate debris. The plates were cut with a notcher (no. 100, Whitney Metal Tool Co.) into 10×10 mm squares. The four edges of each substrate were then ground in the same way as described above. All the substrates were cleaned in ethanol under sonication (Symphony™ Ultrasonic Cleaners, VWR) for 15 minutes. Before coating processes, the Mg substrates were examined using a field emission scanning electron microscopy (SEM; XL30-FEG, Philips) at an accelerating voltage of 15 kV.

### Preparation and Characterization of Nanophase HA

Nanophase HA was synthesized using a wet chemistry precipitation method followed by hydrothermal treatment. Briefly, a 1M calcium nitrate [Ca(NO₃)₂; Sigma Aldrich] solution with a pH of 10 and a 0.6M ammonium hydrogen phosphate [(NH₄)₂HPO₄; Sigma Aldrich] solution with a pH of 11 were prepared separately at 40° C. Ammonium hydroxide (NH₄OH; Sigma Aldrich) was used to adjust the pH of these solutions. Adding calcium nitrate solution drop wise into the ammonium phosphate solution at 40° C. led to precipitation of calcium phosphate (CaP). The mixture was stirred for an additional 20 hours at 40° C., and then centrifuged at 10,000 revolutions per min (RPM). After centrifuge, the supernatant was removed and the pellet at the bottom of the centrifuge tube was re-suspended in an equal volume of deionized (DI) water. The pellet was washed for 5 times by repeating the steps of centrifuge and re-suspending in fresh DI water. After the excess reactants were washed away, the pellet was re-suspended in DI water and hydrothermally treated at 200° C. for 20 hours in an acid digestion bomb (Parr Instrument). After hydrothermal treatment, the nHA was precipitated out of suspension via centrifugation and then dried in vacuum at 80° C. Finally, the dried nHA was ground into a fine powder form using a mortar and pestle. Subsequently, the nHA nanoparticles were used to prepare nHA/PLGA composites for spin coating.

The microstructure and elemental composition of nHA was characterized using a field emission scanning electron microscopy (SEM; XL30-FEG, Philips) and attached energy dispersive x-ray spectroscopy (EDX; EDAX) at an accelerating voltage of 10 kV. The crystal structure of HA was confirmed using x-ray diffraction (XRD; D8 Advance, Bruker AXS), performed at the 35 kV and 20 mA with a 0.02° step size.

Spin Coating the nHA/PLGA Nanocomposites onto the Mg Substrates and the Controls

The nHA/PLGA nanocomposite suspension for spin coating was prepared by first dissolving 0.35 g PLGA (50:50, 40-75 kDa; Sigma Aldrich) in 3 mL chloroform (CHCl₃, Sigma-Aldrich) at 40° C. under low-power sonication (Symphony™ Ultrasonic Cleaner, VWR) for 60 min. After the PLGA was completely dissolved, 0.15 g HA was added into the PLGA solution at 40° C. under low-power sonication for an additional 60 min. The nHA/PLGA suspension was then sonicated using a high power sonicator (9 W, 20 kHz; Misonix Sonicator S-4000) for 10 minutes to improve the dispersion of nHA in PLGA. The nHA/PLGA suspension was then degassed in vacuum at room temperature for 5 min.

The same spin coating procedures were used to deposit nHA/PLGA nanocomposites on Mg, AZ31 and Ti substrates. Before spin coating, the clean substrates were first secured on the spin chuck in the spin coater (PWM32, Headway Research). The nHA/PLGA suspension was applied onto the substrates using a disposable borosilicate glass Pasteur pipette (VWR). The substrates were then spun at 300 RPM for 3 minutes. After spin coating, the substrates were placed in a Teflon dish with the newly coated side facing up, and dried in air at room temperature for 24 hours. The remaining nHA/PLGA composite suspension was transferred into a Teflon dish, dried in air at room temperature for 24 hours, and saved for coating the other side of the substrates. Once the coated side was dried, the saved nHA/PLGA was re-suspended in chloroform at the same concentration and the opposite side of the substrates was spin coated under the same conditions. After spin coating the nHA/PLGA nanocomposites onto the top and bottom surfaces, the four edges were dip coated and dried in air at room temperature for 24 hours. After 24 hours of air dry, the coated substrates were dried in vacuum at room temperature for 48 hours.

The remaining nHA/PLGA solution was cast into a Teflon dish to form a thin film and dried in air for 24 hours followed by vacuum dry for 48 hours at room temperature. The dried nHA/PLGA film was cut into 10×10 mm squares and used as a control for material characterization and degradation experiments.

Spin Coating the PLGA Control onto the Mg Substrates and the Controls

PLGA control coating (without nHA) was deposited onto the Mg, AZ31, and Ti substrates using the similar spin coating procedures as for the nanocomposite coatings. Briefly, PLGA solution was prepared by dissolving 0.45 g PLGA (50:50, 40-75 kDa; Sigma Aldrich) in 3 mL chloroform (CHCl₃; Sigma Aldrich) at 40° C. under low-power sonication (Symphony™ Ultrasonic Cleaner, VWR) for 60 minutes. The top surface of the substrates was spin coated and dried in air for 24 hours. The remaining PLGA solution was saved for spin coating on the bottom surface. The four edges were dip coated with PLGA. The PLGA solution left from spin coating was cast in a Teflon dish and cut into films with a size of 10 mm×10 mm. The PLGA films served as a control for material characterization and degradation experiments.

Depositing CaP Control Coating onto the Mg Substrates and the Controls

The calcium phosphate (CaP) coating was deposited onto the Mg substrates as a ceramic control using an immersion method described by Zhang et al. A concentrated simulated body fluid (SBF) that had three times more Ca²⁺ and HPO₄²⁻ions than 1×SBF, called 3CaP SBF, was used for CaP coating. The prepared substrates were first immersed in 100 mL 3CaP SBF solution at 42° C. for 24 hours, and then dried in air at room temperature. The immersion was repeated with the dried substrates in 100 mL fresh 3CaP SBF solution for another 24 hours. Finally, the CaP coated Mg substrates were rinsed with DI water and dried in air at room temperature. This process led to formation of a low-crystalline apatite coating on the Mg substrates.

### Characterization of the Coatings and Controls

The thicknesses of nHA/PLGA coatings and PLGA coatings on the Mg substrates were measured based on the SEM images of the coating cross-sections using ImageJ. Briefly, the Mg substrates with a thickness of 250 μm were spin coated with PLGA or nHA/PLGA composites by following the same procedures described above. The coated substrates were dried in air for 24 hours and in vacuum for 48 hours at room temperature, cut in half with scissors, and mounted onto a 90-degree sample holder for SEM imaging. The coating thickness was measured at the center of each sample and 1 mm from both edges, and then averaged. The average thickness of PLGA or nHA/PLGA coatings was determined based on three different samples.

The PLGA films, nHA/PLGA films, and their corresponding coatings on the Mg substrates were characterized using the SEM at an accelerating voltage of 5 kV with an original magnification of 100,000×. The CaP coated Mg was examined using the SEM at an accelerating voltage of 15 kV with an original magnification of 2,500×. The dispersion state of nHA in PLGA was determined through quantitative analysis of SEM images using ImageJ. The nHA particles dispersed in the PLGA matrix was first manually outlined and then converted into a binary mask using ImageJ. To quantify nHA dispersion in the PLGA matrix before and after spin coating, the Feret maximum diameter and the areal fraction of the nHA particles in the binary mask were measured and calculated using ImageJ.

### Degradation Studies by Tafel Test

The degradation of the coated versus non-coated Mg samples were tested in revised simulated body fluid (rSBF), which had the same ionic concentration as human blood plasma. Tafel test was conducted according to ASTM standard G 102-89 to predict the degradation rate of the samples. Potentiodynamic polarization curves were generated using a Potentiostat/galvanostat (model 273A; EG&G Princeton Applied Research). The nHA/PLGA coated Mg, PLGA coated Mg, and non-coated Mg samples were tested in triplicate in rSBF at 37° C. Each sample was clamped to the working electrode with half of the samples being immersed in rSBF and the other half above the rSBF solution. An Ag/AgCl reference electrode (part # CHI111, CHI Instruments) and a Pt counter electrode (part # CHI 115, CHI Instruments) were used and immersed in the same rSBF solution. The Tafel test was performed at an electric potential ranging from +1 V to −3 V, with a 10 mV step size and a 0.5-second step time at a 100 mV/s scan rate. On the Tafel plots, straight lines were drawn along the linear portion of the potentiodynamic polarization curves. From the intersection of these straight lines, the corrosion current (I_{corr}) and corrosion potential (E_{corr}) were extrapolated. The corrosion rate (CR) (in mm/year) of the samples was calculated using the following equation from ASTM standard G 102-89. The statistical significance of corrosion rate was determined by Kruskal-Wallis test with an a value of 0.05.

$$CR = \frac{{I}_{corr} \times {K}_{1} \times EW}{ρ \times A} \qquad Eq . 1$$

where I_{CORR} is the corrosion current, K₁ is the constant for unit conversion, EW is the equivalent weight of Mg, ρ is the density of corroding species (Mg), and A is the area of the sample submerged in rSBF. Specifically, K₁=3.27×10⁻³ (mm·g)/(μA·cm·year), EW=12.15, ρ=1.74 g/cm³, A=1.2 cm².

### Degradation Studies by Immersion Method

The degradation of nHA/PLGA coated Mg, PLGA-coated Mg control, CaP-coated Mg control, and non-coated Mg control were further investigated using immersion methods. The PLGA and nHA/PLGA films, their coatings on AZ31 and Ti substrates, and non-coated substrates were used as additional controls. All the samples were weighed, photographed, and disinfected under ultraviolet (UV) radiation before immersion. The samples were first placed into the wells of twelve well tissue culture plates in a laminar flow hood (Model no. NU-425-400, Nuaire), and 3 mL of rSBF was added to each well. The samples were incubated in rSBF under standard cell culture conditions (a sterile, 37° C., 5% CO₂/95% air, and humidified environment) until reaching the prescribed time points. The prescribed time points were 0 hr, 1 hr, 2 hr, 4 hr, 8 hr, 16 hr, and 24 hr to closely mimic the in vivo condition where circulation takes away degradation products regularly. After each time point, the rSBF was removed from the wells and the samples were dried in vacuum for at least 24 hours. The pH of the rSBF was measured using a pre-calibrated pH meter (Symphony SB70P, VWR). The dried samples were weighed and photographed. These samples were then placed into 3 mL of fresh rSBF and incubated for the next prescribed time point. This process was repeated for each prescribed time point. All the samples were handled in sterile conditions during the immersion study.

The rSBF collected at each time point was analyzed for Mg ion concentration ([Mg²⁺]) and Ca ion concentration ([Ca²⁺]) using inductively coupled plasma-atomic emission spectroscopy (ICP-AES; Optima 2000 DV, Perkin Elmer Instruments). Serial diluted MgCl₂ and CaCl₂ solutions were run in parallel to generate standard curves.

After 24 hours of immersion, the delaminated coatings were placed on a conductive copper tape for SEM imaging and EDX analysis. The PLGA coating was imaged at an accelerating voltage of 5 kV with an original magnification of 2500×. The nHA/PLGA coating was imaged at an accelerating voltage of 2 kV with an original of 2500×. EDX analysis was performed at an accelerating voltage of 10 kV with an original magnification of 2500×.

### Results and Discussion

### Characterization nHA Particles

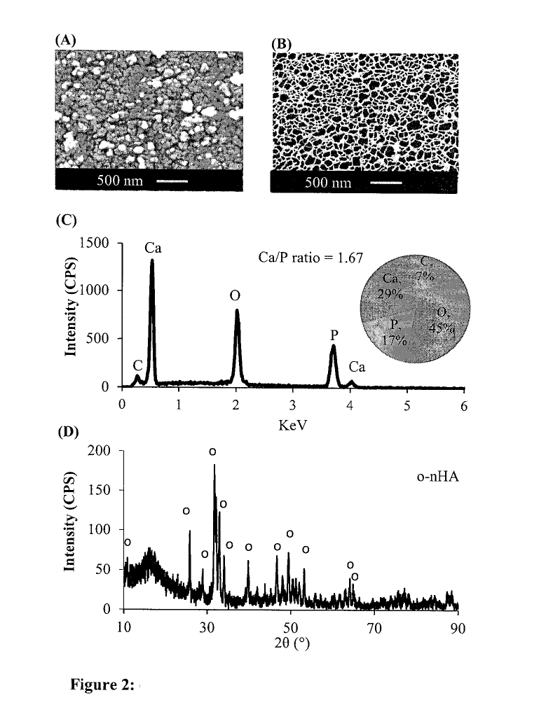

FIG. 2 shows the SEM image, EDX spectrum and quantification of elemental composition, and XRD spectrum of nHA synthesized in these Examples. The nHA particles had an average Feret maximum diameter of 63±50 nm based on quantitative analysis of the SEM images using ImageJ. EDX analysis showed that the nHA particles had a Ca/P ratio of 1.67, which was identical to the Ca/P ratio of natural bone (FIG. 2C). XRD spectrum demonstrated that the synthesized particles had the desired HA phase (FIG. 2D) and similar crystal structure as the HA extracted from bone.

### Characterization of the Coated and Non-coated Mg Substrates

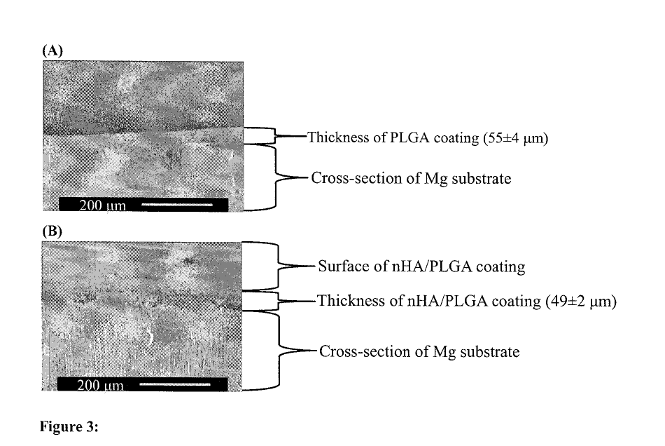

FIG. 3 shows the SEM images of the cross-sections of the PLGA and nHA/PLGA coatings on Mg substrates. The PLGA coating had a thickness of 55±4 μm (FIG. 3A) and the nHA/PLGA coating had a thickness of 49±2 μm (FIG. 3B), according to ImageJ analysis of the cross-section images. The thicknesses of the PLGA and nHA/PLGA coatings were intentionally controlled to be similar to each other during spin coating to ensure the comparability of their degradation results and eliminate the effects of the coating thickness factor on the degradation results.

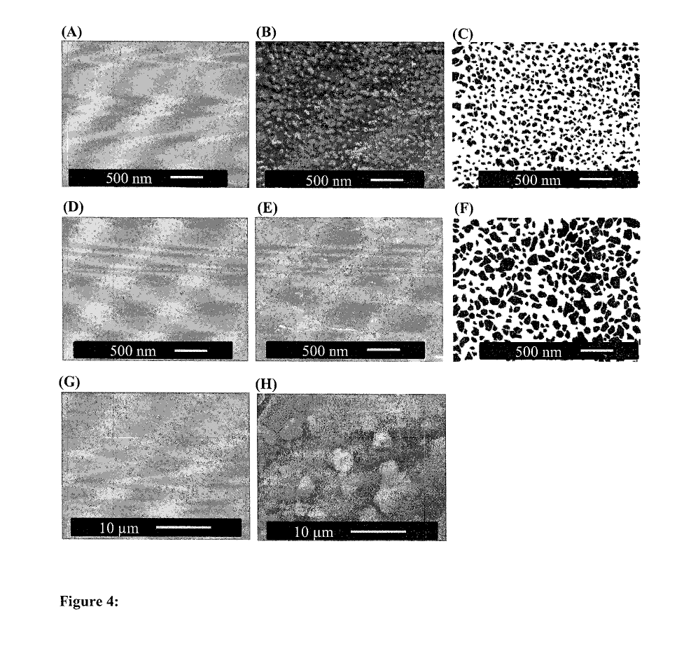

FIG. 4 shows the SEM images of the surfaces of the PLGA and nHA/PLGA films and their respective coatings on the Mg substrates in comparison with non-coated Mg and CaP coated Mg controls. The PLGA film was smooth without any significant surface features (FIG. 4A), while the nHA/PLGA film appeared rough due to the presence of nHA nanoparticles (FIG. 4B). FIG. 4C shows the binary mask of FIG. 4B. Similarly, the PLGA coating on the Mg substrates was smooth (FIG. 4D) and the nHA/PLGA coating appeared rough due to the presence of nHA nanoparticles (FIG. 4E). FIG. 4F shows the binary mask of FIG. 4E. As expected, the non-coated Mg had a smooth surface (FIG. 4G). The CaP coated Mg control had a rough surface with micron-scale CaP particulate features (FIG. 4H).

The nHA particles in the nanocomposite film (FIG. 4B) showed a Feret maximum diameter of 64±28 nm according to quantitative analysis of the ImageJ binary mask (FIG. 4C). This diameter is very similar to the average Feret maximum diameter of nHA particles (i.e., 63±50 nm), which demonstrated the homogenous dispersion of nHA in the PLGA matrix. In comparison, the nHA particles in the nanocomposite coating (FIG. 4E) showed a Feret maximum diameter of 269±130 nm according to quantitative analysis of the ImageJ binary mask (FIG. 4F), which indicated some degree of agglomeration as compared with the nanocomposite film. The degree of nHA agglomeration affected the size scale of the surface features on the nHA/PLGA films and coatings. Even though the size of agglomerates in the nHA/PLGA coating was larger than that of the nHA crystals in deproteinated bone (25-50 nm) and the synthesized nHA particles (63±50 nm), the coating still maintained the surface features at the nano-scale (100 nm to 400 nm) that has been reported to promote osteoblast functions. The agglomerates in the nanocomposite coating might have been caused by the time lapse between the high-power sonication and the spin coating process.

Dispersion of nHA in the nHA/PLGA Film and Coating

The dispersion states of nHA particles in the nanocomposite film or coating play an important role in the mechanical and biological properties of nanocomposites. Specifically, it has been reported that homogenously dispersed nanoparticles in PLGA matrix enhanced mechanical properties of the nanocomposites and improved the osteoblast adhesion and long-term functions in vitro as compared with the agglomerated nanoparticles in PLGA composites. The procedures for the nanocomposite preparation and spin coating had significant effects on the nHA dispersion in the final composite coatings. In this study, the nHA/PLGA composite suspension contained 30 wt. % nHA and 70 wt. % PLGA, corresponding to a 15.39 vol. % nHA and 84.61 vol. % PLGA theoretically, based on the calculation using the following equation.

$${V}_{HA} = \frac{\frac{{M}_{HA}}{ρ HA}}{\frac{{M}_{HA}}{ρ HA} + \frac{{M}_{PLGA}}{ρ PLGA}} \times 100 \% \qquad Eq . 2$$

where V_{HA} is the volume percent of nHA in the nHA/PLGA composites. M_{HA} is the mass of nHA in the composites; ρ_{HA} is the theoretical density of nHA; M_{PLGA} is the mass of PLGA in the composites; ρ_{PLGA} is the theoretical density of PLGA.

The nHA particles occupied 27.90% area on the surface of the nHA/PLGA composite film and 45.30% area on the surface of the nHA/PLGA composite coating on Mg according to the quantitative image analysis of FIGS. 4C and 4F, respectively. The area percentages of nHA particles on the surfaces of the composite film and the coating were both greater than the theoretical volume percentage of nHA particles in the PLGA matrix. This indicated the absence of micron-scale agglomeration because the micron-sized agglomerates would settle down by gravity before the solvent evaporated based on Stoke's Law. As a result, the area percentages of nHA on the surface would decrease if there were micron-scale agglomerates. Additionally, some PLGA might have been trapped in the spaces among the individual nHA particles, which increased the size of particles outlined manually in ImageJ and the calculated nHA area percentages. It is also important to point out that residual PLGA might have adhered to the glassware used for preparing the nanocomposite suspension, which resulted in the decrease of the PLGA volume percent and increase of nHA volume percent in the composite films and coatings. Moreover, initiating the spin coating process immediately after high power sonication of the composite suspensions will further improve the dispersion of nHA in the nanocomposite coatings.

### Degradation Rate Determined by Tafel Test

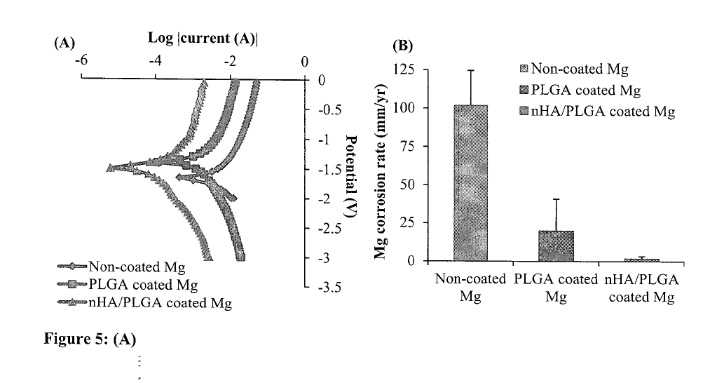

The PLGA and nHA/PLGA coatings significantly increased the corrosion potential (E_{corr}) and decreased the corrosion current (I_{corr}) of Mg substrates during the Tafel test in rSBF at 37° C., as shown in FIG. 5A. Therefore, the Mg corrosion rate (CR, mm/year) decreased, as shown in FIG. 5B and Table 2. The nHA/PLGA coating increased the average E_{corr} more than the PLGA coating, although both of them showed higher average E_{corr} than the non-coated Mg. The PLGA coatings reduced the I_{corr} one order of magnitude lower than non-coated Mg, and the nHA/PLGA coating reduced the I_{corr} one order of magnitude lower than the PLGA coating. Due to the significant reduction of I_{corr}, the calculated corrosion rates for the PLGA coated and nHA/PLGA coated samples significantly decreased. Specifically, the corrosion rates were in the following order from the slowest to the fastest degrading: nHA/PLGA coated Mg<PLGA coated Mg<non-coated Mg. The effects of PLGA and nHA/PLGA coatings on the degradation rates of Mg were statistically significant, as demonstrated by the calculated p value of 0.00367 using the Kruskal-Wallis test.

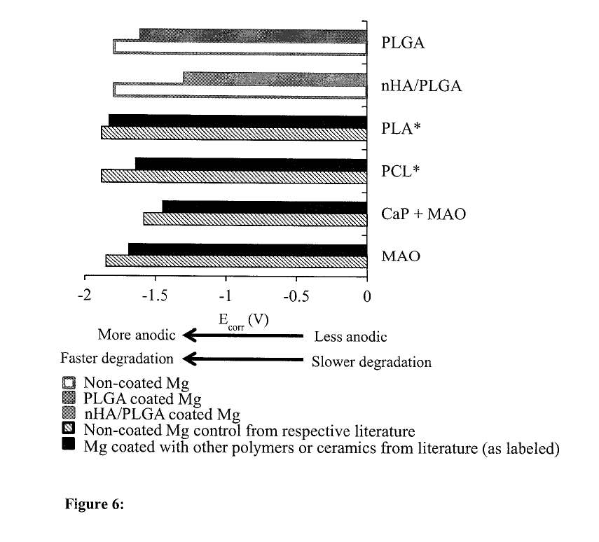

The E_{corr} of the PLGA and nHA/PLGA coated Mg samples was compared with the E_{corr} of Mg substrates coated with other polymers or CaP reported in literature, as shown in FIG. 6. The nHA/PLGA coatings reduced the E_{corr} more effectively than the PLGA coating, poly(lactic acid) (PLA) coating, PCL coating, micro-arc oxidized (MAO) Mg surface, and CaP coating on MAO Mg surface. This demonstrated the promising potential of nHA/PLGA coating for controlling Mg degradation. It is important to mention that the samples were only immersed in rSBF for several minutes during Tafel test, which may not accurately reflect the long-term degradation behavior of the samples. The long-term degradation processes may be affected by ongoing changes to the sample microstructures and other continuous reactions, e.g., the balance of the dissolution reactions versus precipitation of degradation products on the surface.

### Degradation Determined by Immersion Method

Surface Morphology Change during the Sample Degradation in rSBF

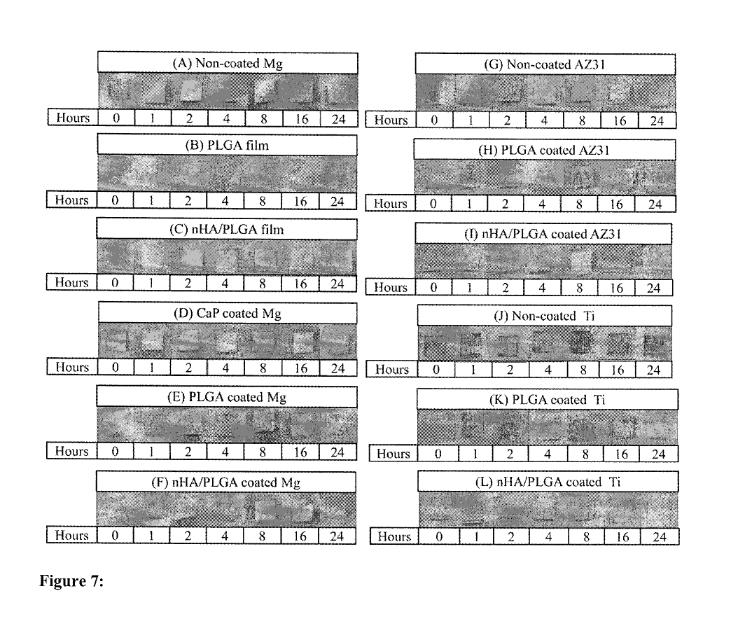

Immersion of the samples in the rSBF solution altered their surfaces, as shown in FIG. 7. The non-coated Mg and AZ31 substrates initially had shiny silver colored and smooth metallic surfaces. After 1 hour incubation in rSBF, their shiny smooth surfaces turned into beige colored rough surfaces due to oxidation of Mg and deposition of degradation products. The non-coated Ti surfaces did not change much after incubation in rSBF, although they were slightly less shiny after 24 hour incubation. The PLGA and nHA/PLGA coatings on the Mg and AZ31 substrates initially were transparent, and their appearance changed dramatically after immersion in the rSBF. Small pores started to form in both of the PLGA and the nHA/PLGA coatings after 1 hour incubation, and then grew larger with formation of more pores as the incubation time increased. With the progression of the degradation, the adjacent pores started to merge with one another to form larger cavities, and the PLGA and nHA/PLGA coatings on both the Mg and AZ31 substrates became opaque white. Eventually, the propagation of the pores caused the coatings to delaminate from the Mg and AZ31 substrates. The nHA/PLGA coatings delaminated earlier than the PLGA coatings from the Mg substrates, after 4 hour and 8 hour incubation, respectively. Similarly, from the AZ31 substrates, the nHA/PLGA coatings delaminated after 4 hour incubation while the PLGA coatings delaminated after 8 hour incubation. The CaP coated Mg control showed similar color throughout the immersion study and visible degradation products started to accumulate on the surface after 4 hour incubation.

The PLGA and nHA/PLGA coatings on the Ti control substrates did not show any significant change throughout the study. The PLGA coatings on the Ti substrates remained transparent, but the nHA/PLGA coatings became slightly opaque after 24 hour incubation. This difference may have been caused by the increased deposition of calcium phosphate minerals onto the nHA/PLGA coated Ti than that onto the PLGA coated Ti. The PLGA and nHA/PLGA coatings on Ti substrates did not delaminate in contrast to their significant delamination from the Mg and AZ31 substrates. When comparing Ti substrates with Mg-based substrates, Ti is inert and non-degradable in rSBF, but Mg and AZ31 are degradable in rSBF and generate H₂ gas. The production of H₂ gas may have been the major cause of the cavity formation at the coating-substrate interface and the eventual coating delamination. The production of H₂ gas also indicated that water penetrated through the coatings to react with Mg-based substrates.

The PLGA and nHA/PLGA films maintained their structural integrality during 24 hour incubation in rSBF. As the immersion time increased, both the PLGA and nHA/PLGA films turned from transparent to opaque with the formation of micropores. The nHA/PLGA films seemed to have more CaP precipitates on the surface than the PLGA film. Interestingly, neither the PLGA nor the nHA/PLGA films exhibited gas cavities during immersion as those shown in the PLGA and nHA/PLGA coated Mg and AZ31 substrates.

### Mass Change of the Samples During Degradation

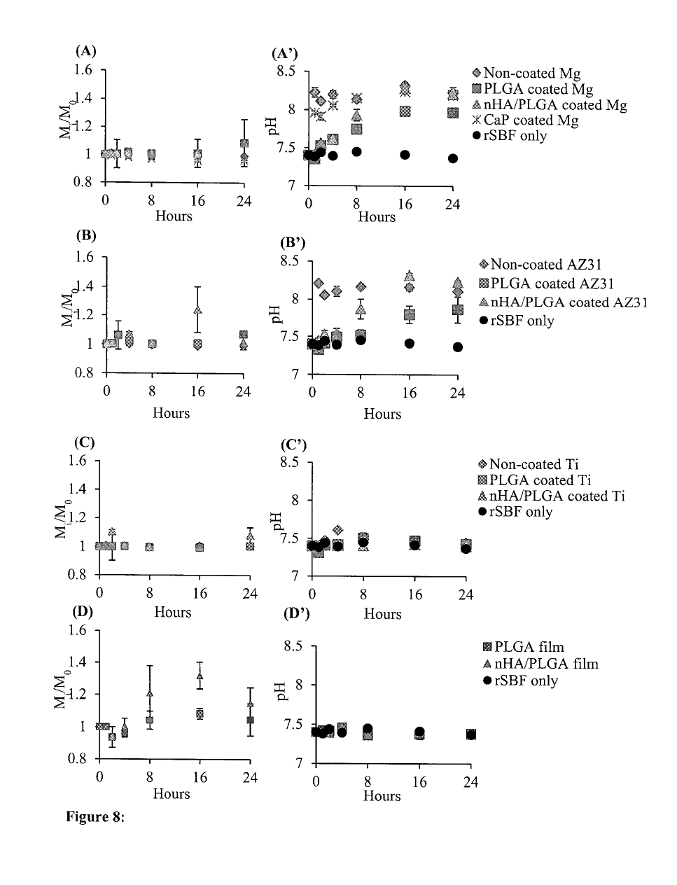

Immersion of the coated and non-coated substrates in the rSBF solution had profound effects on the sample mass change due to the simultaneously occurring degradation, precipitation and deposition processes (FIG. 8 A-D). As shown in FIG. 8A, similar mass changes were observed for all the samples initially, but after 24 hour incubation, the PLGA and nHA/PLGA coated Mg samples showed more mass gain than the non-coated Mg and CaP coated Mg samples. The mass increase of the PLGA and nHA/PLGA coated Mg samples may have been due to accumulation of Mg degradation products, and deposition of CaP salts from rSBF on the surface.

FIG. 8B shows the mass change of the coated and non-coated AZ31 samples. The PLGA coated AZ31 samples showed more mass gain in average than the non-coated AZ31 samples only after 24 hour incubation. The nHA/PLGA coated AZ31 samples showed mass gain after 16 hour incubation. After 24 hour incubation, however, the mass dropped to approximately equivalent to the initial sample mass. This increase and decrease of mass change was observed at 16-24 hour incubation period, because the nHA/PLGA coated AZ31 samples first had significant deposition from the rSBF solution, and then the coating delamination occurred at this period resulted in the release of degradation products from the AZ31 substrates.

FIG. 8C shows the mass change of the coated and non-coated Ti samples. The PLGA coated, nHA/PLGA coated and non-coated Ti maintained similar mass to the initial mass during their immersion in rSBF. This was expected since Ti is inert in rSBF. Moreover, the mass of the Ti substrates was much greater than the mass of the PLGA and nHA/PLGA coatings, which made the relatively small mass changes from the coating degradation or CaP deposition undetectable. FIG. 8D shows the mass change of the PLGA and nHA/PLGA films. The mass of the PLGA and nHA/PLGA films increased due to the deposition of CaP salts from the rSBF onto the films. The nHA/PLGA films gained significantly more mass than the PLGA films because the homogenously dispersed nHA in the films acted as nucleation sites for enhanced CaP deposition from rSBF.

The pH Changes of rSBF During the Sample Degradation

Immersion of the coated and non-coated Mg and AZ31 substrates in the rSBF resulted in pH increase, while the coated and non-coated Ti samples and the films did not show any significant pH changes (FIG. 8 A′-D′). The effects of Mg degradation on the pH changes of the rSBF immersion solution were more pronounced than the mass changes of the samples. FIG. 8A′ shows the pH changes of rSBF when cultured with the coated and non-coated Mg samples in comparison with rSBF control. At the first 8 hour incubation, the pH of rSBF cultured with the non-coated and CaP coated Mg samples increased more significantly than the PLGA coated and nHA/PLGA coated Mg samples, which indicated that the PLGA coating and nHA/PLGA coating alleviated alkalization of the rSBF. After the 8 hour time point, the PLGA coating decreased rSBF alkalization from Mg degradation more effectively than the nHA/PLGA coating because the PLGA coatings delaminated 4 hours later than the nHA/PLGA coatings. As the nHA/PLGA coatings delaminated after 8 hour incubation, the pH steadily increased until it eventually equaled to the pH of the rSBF containing the non-coated and CaP coated Mg samples. Although the pH continued to increase for the rSBF containing the PLGA coated Mg samples, it was still lower than the pH of rSBF containing the non-coated Mg samples.

As compared to FIG. 8A′, FIG. 8B′ shows the similar trend for the PLGA coated and nHA/PLGA coated AZ31 samples versus non-coated AZ31 samples. Briefly, before the 8 hour time point, the PLGA and nHA/PLGA coatings relieved alkalinity of rSBF to some degree as compared with much higher pH for the non-coated AZ31 samples. However, the pH of rSBF containing the PLGA and nHA/PLGA coated AZ31 samples increased after the 8 hour time point, and reached a similar level as the non-coated AZ31 samples at 24 hour time point due to the coating delamination. Delaminated coatings caused rapid release of a large amount of trapped Mg degradation products, which increased the local pH.

In contrast to the Mg-based substrates, the coated and non-coated Ti control samples did not cause any significant pH changes in rSBF, as shown in FIGS. 8C′. Ti is an inert metal in the human body, and thus it is expected that Ti is not degradable in rSBF. FIG. 8D′ shows that the PLGA and nHA/PLGA films did not cause any detectable pH change in rSBF. Considering the degradation mechanisms of PLGA and nHA, the 24 hour incubation period was too short for them to induce any detectable changes to local pH. Previous studies have confirmed this; specifically, Liu et al. showed that the phosphate buffered saline did not show any detectable pH drop until after 3 days of incubation with PLGA.

The Changes of Mg and Ca Ion Concentrations in rSBF During Degradation

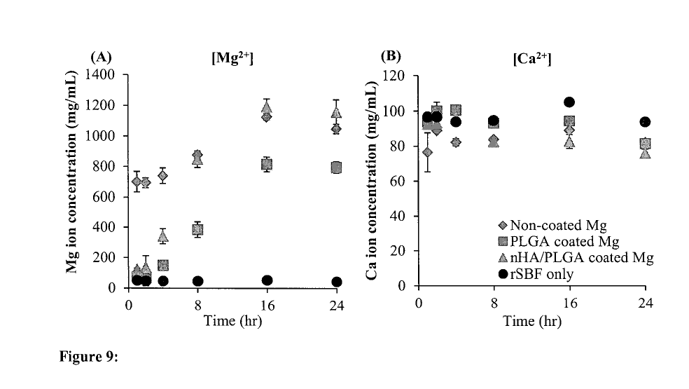

Immersion of PLGA coated Mg, nHA/PLGA coated Mg, and non-coated Mg samples in rSBF altered the Mg and Ca ion concentration of rSBF, as shown in FIG. 9. FIG. 9A shows that Mg ion concentration in rSBF increased for all the samples, including non-coated Mg, PLGA coated Mg, and nHA/PLGA coated Mg, as compared to the constant Mg ion concentration of the rSBF control. Before the 8 hour time point, the non-coated Mg samples released more Mg ions than the PLGA coated and nHA/PLGA coated Mg samples. This indicated that the PLGA and nHA/PLGA composite coatings protected the Mg substrates from degradation. After the 8 hour time point, the Mg ion concentrations of rSBF containing the PLGA and nHA/PLGA coated Mg samples increased significantly. Specifically, at the 24 hour time point, the nHA/PLGA coated Mg samples released a similar amount of Mg ions as the non-coated Mg control due to the coating delamination. Interestingly, the PLGA coated Mg samples released less amounts of Mg ions than the non-coated Mg control throughout the 24 hour incubation period, mainly due to the delayed delamination of the PLGA coating. It is important to mention that the Mg ion concentrations (FIG. 9A) corresponded to the alkaline pH of the rSBF (FIG. 8A′), as both Mg²⁺ and OH⁻ions were released during Mg degradation.

FIG. 9B shows that the PLGA coated Mg, nHA/PLGA coated Mg, and non-coated Mg samples all reduced the concentrations of Ca ions in the rSBF at the 24 hour time point as compared with the rSBF control. The reduction of Ca ion concentration in rSBF was due to the deposition of Ca containing salts onto the samples during incubation. Specifically, after 8 hour incubation, the nHA/PLGA coated Mg samples showed more reduction in Ca ion concentration than the PLGA coated and non-coated Mg samples, indicating more Ca containing salts deposited on the nHA/PLGA coated surfaces.

Surface Morphology and Composition of the Coatings During Immersion in the rSBF

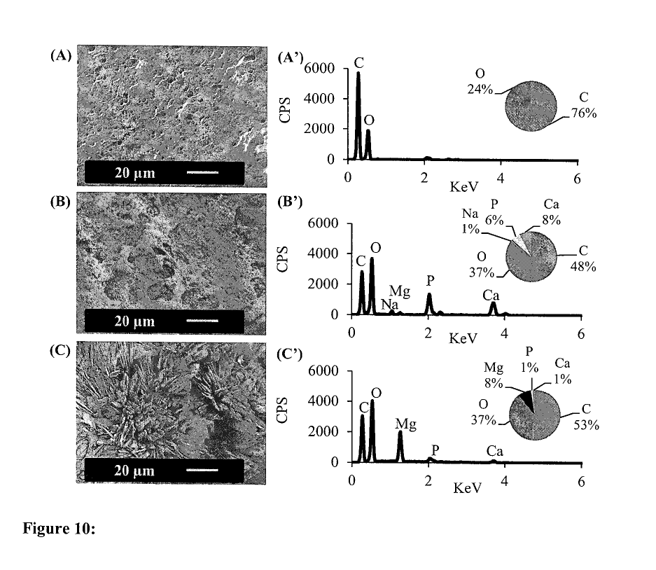

The PLGA and nHA/PLGA coatings on Mg substrates after 24 hour degradation in rSBF were examined using SEM and EDX, as shown in FIG. 10 and Table 3. FIG. 10A shows the presence of micropores on the PLGA coating. These micropores allowed water to penetrate the coating and reach the Mg substrate. According to the EDX results shown in FIG. 10A′, no CaP deposition was observed on the PLGA coatings. The surface morphology of nHA/PLGA coating also changed significantly due to the CaP deposition and PLGA degradation, as shown in FIGS. 10B and 10C. According to the EDX results shown in FIGS. 10B′ and 10C′, a significant amount of CaP deposited onto the nHA/PLGA coatings. The nHA in the PLGA matrix served as the nucleation sites to attract the CaP mineral deposition. The deposition of CaP was also confirmed by the reduction of Ca ion concentration in the rSBF (FIG. 9B).

The deposition of Ca containing minerals onto the surfaces has important implications on the bioactivity of the coatings. PLGA did not attract much CaP deposition in the rSBF, confirming that the bioactivity and osteoconductivity of PLGA are limited. Apatite rosettes with increased Mg concentrations were observed on the nHA/PLGA coating (FIG. 10C), indicating possible Mg substitution of Ca in the CaP crystal structure. Mg substitution of Ca in the HA crystals has been known for enhancing osteoblast adhesion and long-term functions on the HA, and improving osseointegration of the implants coated with HA. Additionally, the incorporation of nHA in the PLGA matrix increased water absorption, which in turn may have resulted in more rSBF constituents available locally for deposition onto the surface.

### Gas Evolution Due to Mg Degradation

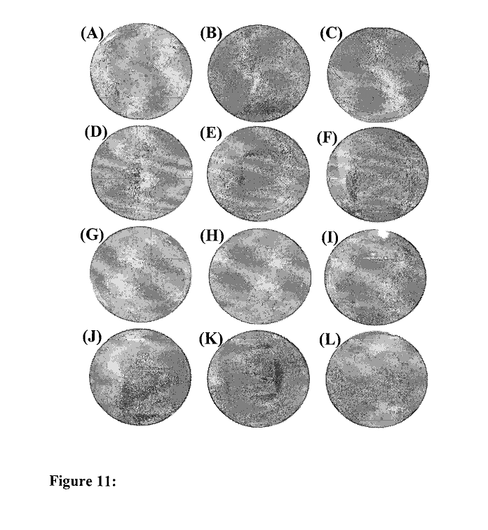

Hydrogen gas (H₂) is another degradation product of Mg in addition to Mg ions and hydroxide ions. FIG. 11 shows that visible gas bubbles formed in the rSBF containing the coated and non-coated Mg and AZ31 samples, but neither in the rSBF containing the coated and non-coated Ti samples nor in the rSBF containing the PLGA and nHA/PLGA films. This indicated that the gas bubbles were from the H₂ produced by the reaction between Mg and water. The rSBF containing the non-coated Mg, the CaP coated Mg, and the non-coated AZ31 samples showed more bubbles than that of the PLGA coated and nHA/PLGA coated Mg and AZ31 samples. Moreover, the H₂ bubbles distributed across the culture wells for the non-coated and CaP coated Mg samples and the non-coated AZ31 samples. In contrast, the gas bubbles were only observed around the edges of the PLGA and nHA/PLGA coated Mg and AZ31 samples. Less gas bubbles around the PLGA coated and nHA/PLGA coated Mg and AZ31 samples indicated that these coatings did protect the substrates from rapid degradation initially.

Although the degradation of PLGA releases carbon dioxide gas (CO₂), the gas bubbles observed in FIG. 11 is unlikely to be CO₂. PLGA has to first degrade into lactic and glycolic acids (intermediate degradation products) in order to release CO₂ and water as the final degradation products. It took several weeks for a PLGA scaffold to degrade to a level when the dissolved lactic acid could be detected. As Ti is inert, there were no bubbles observed around the PLGA and nHA/PLGA coated Ti and non-coated Ti, confirming that the gas bubbles were from the H₂ gas released from Mg degradation.

### Discussion on Degradation Processes and Coating Delamination

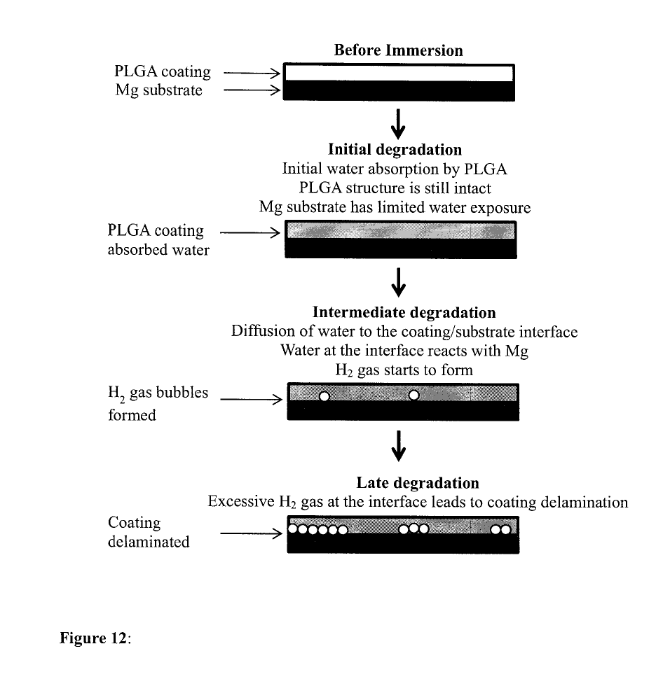

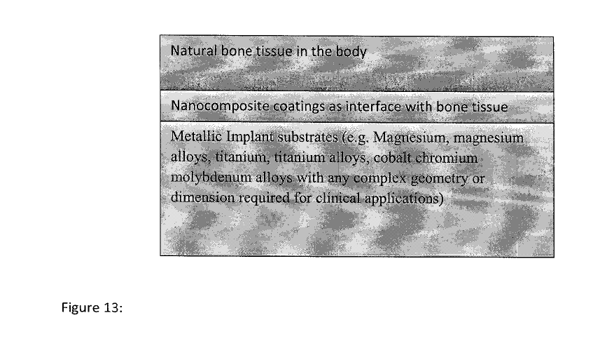

FIG. 12 illustrates the degradation processes of the PLGA and nHA/PLGA coated Mg or AZ31 samples and the speculated mechanisms for the delamination of the coatings. Before the PLGA coated Mg or AZ31 samples were immersed into the rSBF, the PLGA adhered onto the Mg samples uniformly. As soon as the samples were exposed to the rSBF solution, PLGA started to adsorb water, which led to the formation of micropores in the PLGA coating. These micropores propagated as the incubation time in the rSBF increased, which enabled the water to diffuse through the coating to reach the Mg substrates. Once the water reached the interface between the coating and the Mg substrate, Mg started to degrade by reacting with water and release H₂ gas. The accumulation of the H₂ gas at the coating-substrate interface caused the formation of gas cavities, eventually leading to coating delamination. Therefore, water absorption by the PLGA and H₂ gas evolution from the Mg degradation both contributed to the coating delamination.

It is important to point out that the nHA/PLGA coated Mg increased the corrosion potential as compared with the PLGA coated and non-coated Mg, predicting slower degradation. However, the nHA/PLGA coating delaminated earlier than the PLGA coating from the Mg substrates, as the incorporation of nHA into the PLGA matrix might have increased water absorption into the matrix. Transportation processes within a PLGA matrix might be increased by nHA exerting an osmotic swelling pressure, as indicated by the increased dye release from a PLGA-based polymer containing HA. With the increase of water absorption, more water became available at the interface of the coating and Mg substrate, which allowed Mg to degrade more rapidly and form more H₂ gas based on the Reaction 1. The increased production of H₂ gas accelerated the propagation of the gas cavities, which in turn accelerated the previously described delamination processes. Therefore, to improve the efficacy of the nHA/PLGA coating in protecting Mg from rapid degradation, we should fine-tune the coating properties to reduce water absorption during immersion in the rSBF.

The coating delamination can be prevented through reducing water absorption by the polymer. The composition and molecular weight of the polymers play important roles in their water absorption property. Specifically in PLGA, glycolide is hydrophilic and increases water absorption, while lactide is more hydrophobic due to an additional methyl group on its chain. It has been reported that lactide rich PLGA absorbs less water than glycolide rich PLGA. A lactide enriched nHA/PLGA coating may be more effective in reducing water absorption and limiting H₂ evolution from Mg degradation at the interface, thus preventing the coating from delamination.

### CONCLUSION

The nHA and PLGA possess mechanical, biological and degradation properties complementary to Mg alloys, and, thus, the nHA/PLGA nanocomposite is promising as a coating material for Mg and its alloys. The PLGA and nHA/PLGA coatings initially reduced the degradation rates of Mg and AZ31 substrates, beneficial for preventing premature mechanical failure and local alkalinity caused by rapid Mg degradation. Increased CaP deposition was observed on the nHA/PLGA coated Mg substrates after incubation in rSBF, which may improve the bioactivity of the surface for osteoblast adhesion and long-term functions. However, the delamination of PLGA and nHA/PLGA coatings from Mg alloy substrates remains a challenge. Limiting water absorption by the PLGA matrix will prevent delamination and improve the coating efficacy in controlling Mg alloy degradation. The combination of the nHA/PLGA nanocomposites with Mg alloys provides a new multifunctional material for orthopedic implant applications.

It should be understood, of course, that the foregoing relates to exemplary embodiments of the invention and that modifications may be made without departing from the spirit and scope of the invention as set forth in the following claims.

What is claimed is:

1. A biodegradable device, comprising:
a substrate made of a biodegradable metal selected from the group consisting of MgY, AZ alloys, MgCa, MgZn, MgZnCa, MgZnCaZr, MgZnSr, MgLi; and
a nano-ceramic/polymer composite coating on the substrate.

2. A medical device, comprising:
a substrate made of a metal selected from the group consisting of Ti, Ti-6Al-4V, CoCr, and CoCrMo; and
a nano-ceramic/polymer composite coating on the substrate.

3. A biodegradable device, comprising:
a metallic substrate; and
a nano-ceramic/polymer composite coating on the substrate, wherein the coating includes a nano-ceramic and a polymer.
